(19) United States
(12) Patent Application Publication
(10) Pub. No.: US 2023/0355313 A1
(43) Pub. Date: Nov. 9, 2023

(54) SYSTEM AND METHOD FOR HARVESTING AND IMPLEMENTING HAIR GRAFT ON BODY SURFACE WITHOUT USING PRESERVATIVES

(71) Applicant: Gajanan Anand Jadhao, Pune (IN)

(72) Inventor: Gajanan Anand Jadhao, Pune (IN)

(21) Appl. No.: 18/356,669

(22) Filed: Jul. 21, 2023

(51) Int. Cl. A61B 34/10; A61B 17/3205; A61F 2/10; A61F 2/00

(52) U.S. Cl. CPC A61B 34/10; A61B 17/32053; A61F 2/10; A61F 2/0095; A61B 2034/105; A61B 2034/104; A61B 2017/00057

(57) ABSTRACT

The present invention relates to a field of medical and hair transplant. More particularly, the present invention relates to a system and a method for harvesting and implementing hair graft on body surface. The system includes a harvester pointer communicatively connected with the processor, wherein the processor configured to instruct the harvester pointer to trace the marked points in the first area of the body surface of the subject and harvest the hairs through plunging mechanism; and an implanter pointer connected with the trimmer and communicatively connected with the processor, wherein the processor configured to instruct the implanter pointer to catch the trimmed hair graft from the trimmer and implant the hair graft at a particular marked point in the second area of the body surface of the subject through injection mechanism.

### FIELD OF THE INVENTION

The present invention relates to a field of medical and hair transplant. More particularly, the present invention relates to a system and a method for harvesting and implementing hair graft on body surface.

### BACKGROUND OF THE INVENTION

During medical operative interventions in a patient, especially if the intervention has an important temporal duration, it is inevitable that movements and/or interruptions will occur on the part of the patient. These interruptions may be mechanical, electrical, hardware, software or medical in nature, or be caused by some other means. For example, it may be convenient or simply unavoidable that the patient changes position during the intervention, or that the patient and/or the doctor temporarily leave the place where the operation is being performed before resuming it. For various medical interventions, including aesthetic interventions, interventions, and in particular, for example, in the case of a patient undergoing an intervention of 20 hair transplants, it is relevant to have follicular units extracted from a donor area (for example, in the patient's scalp) for transplantation, or having follicular units implanted in a recipient area (for example, a baldness area in the patient's scalp). Performing these interventions normally takes several hours or more. In some aspects, the patient may remain in the intervention chair but needs to change posture due to discomfort and/or fatigue, or simply moves due to breathing or other natural movements. In other aspects the patient may have to interrupt the intervention to temporarily leave the chair.

The main goal of designing Real-Time FUE was to reduce the graft harvesting time, avoid chemicals to preserve grafts before implantation, and cut-off the high cost as charged in the DHI and other hair transplant method.

The DHI method mostly causes inconvenience to clients because of inefficient results and its costs. The drawbacks of DHI were mainly higher cost, poor results, longer duration of surgery, long learning curve and patient discomfort. These above mentioned drawbacks are excluded in RT FUE.

In Real-Time FUE, an applicator first draw natural looking hairline, Then slit making which decides patient's hair density, the direction of the hair follicle, and hair angle. This is achieved by making slits first to get an idea of the number of grafts needed to achieve the desired hair density. RT FUE involves concurrent extraction and implantation of follicles. Hence, it helps increase the follicle survival rate by preventing preservatives' use for storing hair follicles until implantation, as done in DHI and other technique. Thus, it is known as Real-Time FUE as there is no delay between extraction and implantation step.

Conventional FUE is a standard, step-by-step process where the grafts are stored in a preservative solution after extraction. After slit making, the Implanter implants the preserved grafts in your scalp. In conventional FUE, a qualified hair transplant surgeon decides the graft requirement, which is not accurate and exact, but it's always approximate.

Therefore, there exists a need to provide a system and method for harvesting and implementing hair graft on body surface. In the claimed method, there is no need to preserve the grafts. The graft extraction, graft harvesting, and implantation steps are done simultaneously. Hence, the grafts are implanted within a few minutes of their harvesting.

The technical advancements disclosed by the present invention overcomes the limitations and disadvantages of existing and convention systems and methods.

### SUMMARY OF THE INVENTION

The present invention relates to a field of medical and hair transplant. More particularly, the present invention relates to a system and a method for harvesting and implementing hair graft on body surface.

In an embodiment, a system for harvesting and implementing hair graft on body surface is provided. The system includes an image capturing unit configured to capture a first image and second image of body surface of a subject, wherein the first image of the body surface refers to a first area where the hairs get harvested and the second image of the body surface refers to a second area where the harvested hairs get implemented; a processor configured to process the captured first and second image of the body surface of the subject, wherein the processor is further configured to mark a plurality of points in the first and second image of the body surface of the subject for harvesting and implementing the hair graft; a plurality of marker communicatively connected with the processor, wherein the processor configured to instruct the plurality of marker to trace the marked points of first and second image of the body surface of the subject and mark a plurality of points in the first area and the second area of the body surface of the subject where hairs get harvested and implemented; a harvester pointer communicatively connected with the processor, wherein the processor configured to instruct the harvester pointer to trace the marked points in the first area of the body surface of the subject and harvest the hairs through plunging mechanism; a trimmer communicatively connected with the processor and the harvester pointer, wherein the trimmer configured to catch harvested hair from the harvester pointer and trim the harvested hair as per the instructions provided by the processor on basis of depth of the marked point in the second area of the body surface of the subject; and an implanter pointer connected with the trimmer and communicatively connected with the processor, wherein the processor configured to instruct the implanter pointer to catch the trimmed hair graft from the trimmer and implant the hair graft at a particular marked point in the second area of the body surface of the subject through injection mechanism.

In another embodiment, method for harvesting and implementing hair graft on body surface, said method includes of capturing, by an image capturing unit, a first image and second image of body surface of a subject, wherein the first image of the body surface refers to a first area where the hairs get harvested and the second image of the body surface refers to a second area where the harvested hairs get implemented; processing, by a processor, the captured first and second image of the body surface of the subject, wherein the processor is further configured to mark a plurality of points in the first and second image of the body surface of the subject for harvesting and implementing the hair graft; tracing, by a plurality of marker, the marked points of first and second image of the body surface of the subject and marking a plurality of points in the first area and the second area of the body surface of the subject where hairs get harvested and implemented; tracing, by a harvester pointer, the marked points in the first area of the body surface of the subject and harvesting the hairs through plunging mechanism; catching, by a trimmer, harvested hair from the harvester pointer and trimming the harvested hair as per the instructions provided by the processor on basis of depth of the marked point in the second area of the body surface of the subject; and catching, by an implanter pointer, the trimmed hair graft from the trimmer and implanting the hair graft at a particular marked point in the second area of the body surface of the subject through injection mechanism.

An object of the present invention is to provide a system and a method for harvesting and implementing hair graft on body surface without using any preservatives;

Another objective of the present invention is to provide graft extraction, graft harvesting, and implantation process simultaneously.

Another objective of the present invention is to provide a “REAL-TIME NON-TOUCH FUE” which aims to reduce the time between harvesting of graft and implantation of the graft.

Another objective of the present invention is to provide a method overcomes the drawback of preservation of grafts as grafts survival decreases with more outer body time.

Another objective of the present invention is to provide to provide a Real-Time Non-touch FUE method does save a great deal of cost. (Costly preservative medium). Since the extracted follicles are directly implanted in the bald area, this method saves a great deal of time and effort.

Another objective of the present invention is to provide a quality of grafts is retained as there is no time gap between the two steps i.e. extraction (harvesting) and implantation. No graft preservation is required and these are directly implanted maintain its cellular integrity, prevents anaerobic metabolism.

Another objective of the present invention is to provide a comparatively quicker and long lasting results. Pre-planning gives an idea about how much grafts are to be extracted rather than taking leap of faith like regular FUE.

Another objective of the present invention is to provide a Continuous non-contact FUE that is a technique for relocating hair follicles without putting them in a protecting arrangement.

Another objective of the present invention is to provide provide a strategy, the hair follicles are removed and embedded at the same time without keeping them in a protecting arrangement, bringing about an ordinary FUE hair relocate.

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present invention will become better understood when the following detailed description is read with reference to the accompanying drawings in which like characters represent like parts throughout the drawings, wherein:

FIG. 1 illustrates a block diagram of a system for harvesting and implementing hair graft on body surface; and

FIG. 2 illustrates a flow diagram of process for harvesting and implementing hair graft on body surface.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present invention will be described below in detail with reference to the accompanying drawings.

The claimed method aims to reduce the time between harvesting of graft and implantation of the graft. This method overcomes the drawback of preservation of grafts as grafts survival decreases with increase in a outer body time. The Real-Time Non-touch FUE method does save a great deal of cost. (Costly preservative medium). Since the extracted follicles are directly implanted in the bald area, this method preserve the quality of grafts as there is no time delay between the two steps i.e. extraction (harvesting) and implantation. No graft preservation is required and these are directly implanted maintain its cellular integrity, prevents anaerobic metabolism in grafts. It provides comparatively quicker and long lasting results. Pre-planning gives an idea about how much grafts are to be extracted rather than taking leap of faith like regular FUE. Continuous non-contact FUE is a technique for relocating hair follicles without putting them in a protecting arrangement. Under this strategy, the hair follicles are removed and embedded at the same time without keeping them in a protecting arrangement, bringing about an ordinary FUE hair relocate.

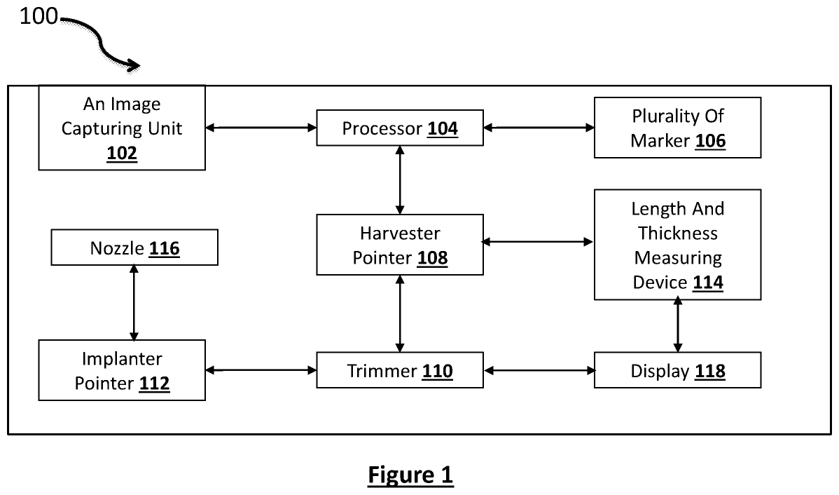

FIG. 1 illustrates a block diagram of a system for harvesting and implementing hair graft on body surface. The system 100 includes an image capturing unit 102 configured to capture a first image and second image of body surface of a subject, wherein the first image of the body surface refers to a first area where the hairs get harvested and the second image of the body surface refers to a second area where the harvested hairs get implemented;

In an embodiment, the system 100 includes a processor 104 configured to process the captured first and second image of the body surface of the subject, wherein the processor 104 is further configured to mark a plurality of points in the first and second image of the body surface of the subject for harvesting and implementing the hair graft;

In another embodiment, the system 100 includes a plurality of marker 106 communicatively connected with the processor 104, wherein the processor 104 configured to instruct the plurality of marker to trace the marked points of first and second image of the body surface of the subject and mark a plurality of points in the first area and the second area of the body surface of the subject where hairs get harvested and implemented;

In another embodiment, the system 100 includes a harvester pointer 108 communicatively connected with the processor 104, wherein the processor 104 configured to instruct the harvester pointer 108 to trace the marked points in the first area of the body surface of the subject and harvest the hairs through plunging mechanism;

In another embodiment, the system 100 includes a trimmer 110 communicatively connected with the processor 104 and the harvester pointer 108, wherein the trimmer 110 configured to catch harvested hair from the harvester pointer and trim the harvested hair as per the instructions provided by the processor on basis of depth of the marked point in the second area of the body surface of the subject; and

In another embodiment, the system 100 includes an implanter pointer 112 connected with the trimmer 110 and communicatively connected with the processor 104, wherein the processor 104 configured to instruct the implanter pointer 112 to catch the trimmed hair graft from the trimmer and implant the hair graft at a particular marked point in the second area of the body surface of the subject through injection mechanism.

In another embodiment, the plurality of marker 106 configured to measure depth of the location of the marked points in the second area of the body surface of the subject.

In another embodiment, the system 100 includes comprising a length and thickness measuring device 114 connected with the processor 104, plurality of marker 106 and harvester pointer 108, wherein the measuring device 114 configured to measure length and thickness of the harvested hair from the first area of the body surface of the subject.

In another embodiment, the trimmer communicatively connected with the plurality of marker 106 and length and thickness measuring device 114, wherein the trimmer configured to trim the harvested hair as per the depth measured by the plurality of marker.

In another embodiment, the plurality of marker 106 configured to mark same numbers in the first area and the second area of the body surface of the subject for harvesting and implementing the hair.

In another embodiment, the harvester pointer 108 configured to harvest the hair on a particular number marked by the plurality of marker in the first area and the implanter pointer configured to implant the same harvested hair on a same number marked in the second area after trimming the harvested hair.

In another embodiment, the system 100 includes a nozzle 116 connected with the implanter pointer configured to provide oxygen to the implanter pointer for implanting the hair graft through injecting mechanism, wherein the nozzle is connected with an oxygen cylinder through a hose.

In another embodiment, the system 100 includes a display 120 configured to provide visual representation of the complete harvesting process and implanting process.

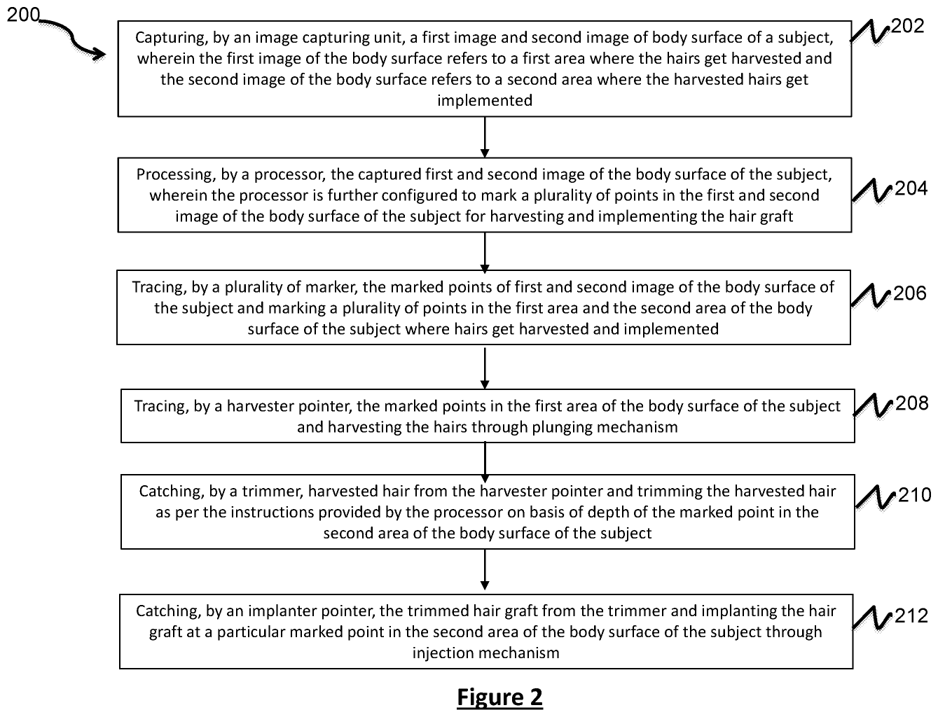

FIG. 2 illustrates a flow diagram of method 200 for harvesting and implementing hair graft on body surface. The method 200 includes

Step 202 of capturing, by an image capturing unit, a first image and second image of body surface of a subject, wherein the first image of the body surface refers to a first area where the hairs get harvested and the second image of the body surface refers to a second area where the harvested hairs get implemented;

Step 204 of processing, by a processor, the captured first and second image of the body surface of the subject, wherein the processor is further configured to mark a plurality of points in the first and second image of the body surface of the subject for harvesting and implementing the hair graft;

Step 206 of tracing, by a plurality of marker, the marked points of first and second image of the body surface of the subject and marking a plurality of points in the first area and the second area of the body surface of the subject where hairs get harvested and implemented;

Step 208 of tracing, by a harvester pointer, the marked points in the first area of the body surface of the subject and harvesting the hairs through plunging mechanism;

Step 210 of catching, by a trimmer, harvested hair from the harvester pointer and trimming the harvested hair as per the instructions provided by the processor on basis of depth of the marked point in the second area of the body surface of the subject; and

Step 212 of catching, by an implanter pointer, the trimmed hair graft from the trimmer and implanting the hair graft at a particular marked point in the second area of the body surface of the subject through injection mechanism.

In an embodiment, the method 200 includes of measuring, by a length and thickness measuring device, length and thickness of the harvested hair from the first area of the body surface of the subject.

Every Hair Transplant Surgery consists of the following steps initially, which are mandatory: Consultation, Hairline Designing, IV Antibiotics, and Anaesthesia.

### Step 1: Slit Making

RT-FUE is a pre-planned procedure. Our surgeon initially performs slit making after completing hairline designing, IV antibiotics, and administering anaesthesia to determine the exact number of grafts extracted for implantation to get the desired density.

### Step 2: Graft Extraction, Graft Harvesting, and Implantation

1. It is a simultaneous process. After slit making, an experienced hair transplant surgeon immediately performs Graft Extraction.

2. Concurrently, the harvested grafts are passed on to a helper to ensure they are healthy and clean. Further, the device passes the grafts to an Implanter, who then immediately implants the same.

3. Implantation is done within a few minutes by the Non-touch technique. The team performing and assisting the surgery must be highly professional and experienced.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

We claim:

1. A system for harvesting and implementing hair graft on body surface, said system comprising:
an image capturing unit configured to capture a first image and second image of body surface of a subject, wherein the first image of the body surface refers to a first area where the hairs get harvested and the second image of the body surface refers to a second area where the harvested hairs get implemented;
a processor configured to process the captured first and second image of the body surface of the subject, wherein the processor is further configured to mark a plurality of points in the first and second image of the body surface of the subject for harvesting and implementing the hair graft;
a plurality of marker communicatively connected with the processor, wherein the processor configured to instruct the plurality of marker to trace the marked points of first and second image of the body surface of the subject and mark a plurality of points in the first area and the second area of the body surface of the subject where hairs get harvested and implemented;
a harvester pointer communicatively connected with the processor, wherein the processor configured to instruct the harvester pointer to trace the marked points in the first area of the body surface of the subject and harvest the hairs through plunging mechanism;
a trimmer communicatively connected with the processor and the harvester pointer, wherein the trimmer configured to catch harvested hair from the harvester pointer and trim the harvested hair as per the instructions provided by the processor on basis of depth of the marked point in the second area of the body surface of the subject; and
an implanter pointer connected with the trimmer and communicatively connected with the processor, wherein the processor configured to instruct the implanter pointer to catch the trimmed hair graft from the trimmer and implant the hair graft at a particular marked point in the second area of the body surface of the subject through injection mechanism.

2. The system as claimed in claim 1, wherein the plurality of marker configured to measure depth of the location of the marked points in the second area of the body surface of the subject.

3. The system as claimed in claim 1, further comprising a length and thickness measuring device connected with the processor, plurality of marker and harvester pointer, wherein the measuring device configured to measure length and thickness of the harvested hair from the first area of the body surface of the subject.

4. The system as claimed in claims 2 and 3, wherein the trimmer communicatively connected with the plurality of marker and length and thickness measuring device, wherein the trimmer configured to trim the harvested hair as per the depth measured by the plurality of marker.

5. The system as claimed in claim 1, wherein the plurality of marker configured to mark same numbers in the first area and the second area of the body surface of the subject for harvesting and implementing the hair.

6. The system as claimed in claim 5, wherein the harvester pointer configured to harvest the hair on a particular number marked by the plurality of marker in the first area and the implanter pointer configured to implant the same harvested hair on a same number marked in the second area after trimming the harvested hair.

7. The system as claimed in claim 1, further comprising a nozzle connected with the implanter pointer configured to provide oxygen to the implanter pointer for implanting the hair graft through injecting mechanism, wherein the nozzle is connected with an oxygen cylinder through a hose.

8. The system as claimed in claim 1, further comprising a display configured to provide visual representation of the complete harvesting process and implanting process.

9. A process for harvesting and implementing hair graft on body surface, said process comprising:
capturing, by an image capturing unit, a first image and second image of body surface of a subject, wherein the first image of the body surface refers to a first area where the hairs get harvested and the second image of the body surface refers to a second area where the harvested hairs get implemented;
processing, by a processor, the captured first and second image of the body surface of the subject, wherein the processor is further configured to mark a plurality of points in the first and second image of the body surface of the subject for harvesting and implementing the hair graft;
tracing, by a plurality of marker, the marked points of first and second image of the body surface of the subject and marking a plurality of points in the first area and the second area of the body surface of the subject where hairs get harvested and implemented;
tracing, by a harvester pointer, the marked points in the first area of the body surface of the subject and harvesting the hairs through plunging mechanism;
catching, by a trimmer, harvested hair from the harvester pointer and trimming the harvested hair as per the instructions provided by the processor on basis of depth of the marked point in the second area of the body surface of the subject; and
catching, by an implanter pointer, the trimmed hair graft from the trimmer and implanting the hair graft at a particular marked point in the second area of the body surface of the subject through injection mechanism.

10. The process as claimed in claim 9, further comprising measuring, by a length and thickness measuring device, length and thickness of the harvested hair from the first area of the body surface of the subject.
